(19) United States
(12) Patent Application Publication
(10) Pub. No.: US 2015/0369825 A1
(43) Pub. Date: Dec. 24, 2015

(54) Diagnosis of Alzheimer Disease, Cognitive Decline and Dementia

(71) Applicants: Sophia SCHEDIN WEISS, (US); Lars TJERNBERG, (US); Bengt WINBLAD, (US)

(72) Inventors: Sophia Schedin Weiss, Sollentuna (SE); Lars Tjemberg, Stockholm (SE); Bengt Winblad, Enskede (SE)

(21) Appl. No.: 14/770,295

(22) PCT Filed: Feb. 26, 2013
(86) PCT No.: PCT/SE2013/050173

§ 371 Date: Aug. 25, 2015

(51) Int. Cl. G01N 33/68

(52) U.S. Cl. CPC G01N 33/6896; G01N 2400/00; G01N 2440/38; G01N 2800/2814; G01N 2800/2821; G01N 2570/00; G01N 2800/52

(57) ABSTRACT

The present embodiments relate to the usage of selected protein-derived glycans as biomarkers for diagnosis of Alzheimer disease, cognitive decline and dementia.

### TECHNICAL FIELD

The present embodiments generally relate to the medical field, and in particular to diagnosis of Alzheimer disease, cognitive decline and dementia.

### BACKGROUND

Alzheimer disease (AD) is the main cause of dementia, affects around 30 million people and the number is growing as life expectancy increases around the world. AD is a progressive neurodegenerative disease which affects first short-term memory and proceeds to loss of cognitive functions and dementia. Mild cognitive impairment (MCI) is an intermediate stage of dementia and approximately half of the MCI cases will convert into AD within a three-year period. Current AD treatments only give symptomatic relief and there is still no cure against AD. Thus, there is an intense research aimed at finding a disease modifying drug. Most evidence suggests that neurotoxic species formed by the amyloid-beta (Abeta) peptide are crucial for AD-pathogenesis, and another hallmark of the disease is the accumulation of so called neurofibrillary tangles formed by the microtubule-associated protein tau. The studies on Abeta have focused on a 40 residues variant (Abeta40) that is produced at high levels, (around 80-90% of all Abeta), and a longer and more hydrophobic variant (Abeta42), which has a strong tendency to polymerize into oligomers and fibrils. Most familial mutations result in increased Abeta42 levels or an increased Abeta42/Abeta40 ratio, and leads to an early onset AD.

During the last years, many clinical trials, e.g. using γ-secretase inhibitors, have failed. However, it is possible that these drugs could still be effective if given at an early stage of the disease. Thus, the possibility to give an early and more accurate diagnosis is important for the outcome of future clinical trials and treatment strategies. At present, a definitive diagnosis of AD can only be made by post-mortem analysis of the brain. Conclusively, there is a need for very early, preferable presymptomatic, biomarkers.

The search for a specific biomarker for AD, a marker that can be easily measured in a large population and diagnose with high sensitivity and specificity, or preferably, predict the disease before clinical symptoms occur, has been ongoing for a long time. So far, no such biomarker has been found. There are a number of commercial assays available, as well as several research efforts still in laboratory phase.

The Abeta/tau ratio in cerebrospinal fluid (CSF) is currently the biomarker for AD with best specificity and sensitivity. There is, for instance, a commercial assay from Innogenetics, INNO-BIA AlzBio3, which combines Abeta42 with total tau and phosphotau. The test is only for research so far and not for diagnostic procedure in a clinical setting.

Abeta oligomers have been shown to be neurotoxic and are hypothesized to act as “seeds” for the formations of plaques, and therefore specific assays have been developed for measuring oligomers present in body fluids, for instance by IBL International, Singulex, Merck and Amorfix.

A blood test with a panel of biomarkers tested in the Alzheimer Disease Neuroimaging Initiative (ADNI)/Australian Imaging Biomarker and Lifestyle study (AIBL) population (Doecke 2012) reported high sensitivity and specificity in AD vs controls. It is unclear if other dementias can be distinguished and if the difference can be seen presymptomatically. Similar results were observed by O'Bryan et al in ADNI population, also a span of 30 proteins (O'Bryan, 2011) and several other research groups have similar approaches in evaluating a panel of biomarkers.

Many studies have been done to explore different types of neuroimaging as possible biomarkers for AD. The most successful technique so far is Positron Emission Tomography (PET), where two approaches dominate:

Fluoro Deoxy Glucose (FDG) PET—measures glucose metabolism in the brain. This is a common tool in the clinical diagnosis of AD but not specific enough to qualify as a single biomarker.
Amyloid PET—images amyloid deposits in the brain. An assessment of brain amyloid by PET can potentially be a tool for determining diagnosis in the clinical setting and for selection of patients for clinical trials. It could add specificity to trials by aiding the division of patients into subgroups. There are several amyloid PET ligands on the market; PM (Pittsburgh Compound B) is the most common, but other ligands have recently been developed by other companies (e.g. EliLilly/Avid, AstraZeneca, GE and Bayer).

PET imaging requires access to PET camera and specifically trained personnel, is invasive, gives a high radiation dose and is not available to all.

Magnetic resonance imaging (MRI) is another neuroimaging technique, which also has two main approaches:

Volumetric MM—this measures the volume of the brain, and is more a method for confirming an established AD diagnosis and follow-up of disease progression in later stages, rather than an early marker. Volumetric MRI is commonly used in the clinical setting today.
Functional MRI—is a collective name for several methods measuring blood flow in the brain. Not many studies have been done but this may be an evolving research area.

There are a number of commercially available tools for cognitive testing. Many of these are used as part of the diagnostic toolkit in the clinical setting today, but so far none of the tests has proven good enough to serve as a single biomarker for the detection of early AD.

In conclusion, there is not yet any single biomarker that can be used to diagnose AD and there is not yet any biomarker that can be used to detect AD sufficiently early, preferably presymptomatically. Thus, there is clearly a need for such a biomarker that can be used for early diagnosis of AD.

WO 2009/150172 relates to an analysis of the protein glycan profile in the blood and the identification of specific peak ratios when compared to healthy aged matched subjects that are typical for AD.

WO 2012/0560008 discloses diagnosis of AD based on the glycosylation pattern of amyloid-beta peptides in body fluids and tissues.

US 2011/0076704 relates to the usage of an α2,6-sialyl residue containing glycoprotein from a human-derived sample in the diagnosis of AD.

### SUMMARY

It is a general objective to provide a biomarker that can be used for diagnosis of Alzheimer's disease, cognitive decline and/or dementia.

It is a particular objective to enable early detection of Alzheimer's disease, cognitive decline and/or dementia in a subject.

It is another particular objective to support evaluation of treatment effects of Alzheimer's disease, cognitive decline and/or dementia.

These and other objectives are met by embodiments disclosed herein.

An aspect of the embodiments relates to an in vitro method of predicting an Alzheimer's disease status, a cognitive decline status and/or a dementia status in a subject. The in vitro method comprises determining, in a biological sample obtained from the subject, an amount of at least one multi-antennary protein-derived N-glycan in which each antenna is capped with sialic acid. The method also comprises predicting the Alzheimer's disease status, the cognitive decline status and/or the dementia status in the subject based on the amount of the at least one multi-antennary protein-derived N-glycan.

Another aspect of the embodiments relates to an in vitro method for evaluating treatment effects of Alzheimer's disease, cognitive decline and/or dementia. The in vitro method comprises determining, in a biological sample obtained from a subject treated for Alzheimer's disease, cognitive decline and/or dementia, an amount of at least one multi-antennary protein-derived N-glycan in which each antenna is capped with sialic acid. The method also comprises evaluating treatment effects of a treatment against Alzheimer's disease, cognitive decline and/or dementia applied to the subject based on the amount of the at least one multi-antennary protein-derived N-glycan.

A further aspect of the embodiments relates to an in vitro method of predicting an Alzheimer's disease status, a cognitive decline status and/or a dementia status in a subject. The method comprises determining, in a biological sample obtained from the subject, an amount of at least one multi-antennary protein-derived N-glycan selected from a group consisting of glycan M, glycan R, glycan T, glycan U and glycan V:

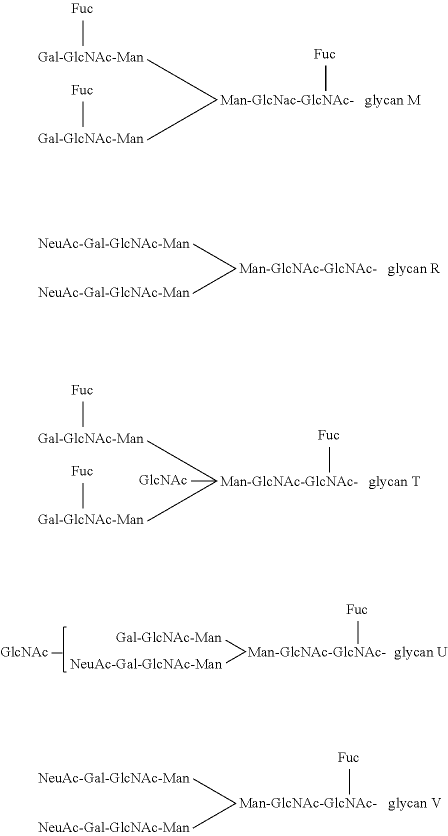

The method also comprises predicting the Alzheimer's disease status, the cognitive decline status and/or the dementia status in the subject based on the amount of the at least one multi-antennary protein-derived N-glycan.

Yet another aspect of the embodiments relates to an in vitro method for evaluating treatment effects of Alzheimer's disease, cognitive decline and/or dementia. The in vitro method comprises determining, in a biological sample obtained from a subject treated for Alzheimer's disease, cognitive decline and/or dementia, an amount of at least one multi-antennary protein-derived N-glycan selected from a group consisting of glycan M, glycan R, glycan T, glycan U and glycan V. The method also comprises evaluating treatment effects of a treatment against Alzheimer's disease, cognitive decline and/or dementia applied to the subject based on the amount of the at least one multi-antennary protein-derived N-glycan.

A further aspect of the embodiments relates to a kit for performing an in vitro method according to above.

Yet another aspect of the embodiments relates to a kit for predicting an Alzheimer's disease status, a cognitive decline status and/or a dementia status in a subject. The kit comprises means for determining, in a biological sample obtained from the subject, an amount of at least one multi-antennary protein-derived N-glycan in which each antenna is capped with sialic acid. The kit also comprises instructions for predicting the Alzheimer's disease status, the cognitive decline status and/or the dementia status in the subject based on the amount of the at least one multi-antennary protein-derived N-glycan.

Another aspect of the embodiments relates to a kit for evaluating treatment effects of Alzheimer's disease, cognitive decline and/or dementia. The kit comprises means for determining, in a biological sample obtained from a subject treated for Alzheimer disease, cognitive decline and/or dementia, an amount of at least one multi-antennary protein-derived N-glycan in which each antenna is capped with sialic acid. The kit also comprises instructions for evaluating treatment effects of a treatment against Alzheimer's disease, cognitive decline and/or dementia applied to the subject based on the amount of the at least one multi-antennary one protein-derived N-glycan.

Yet another aspect of the embodiments relates to a kit for predicting an Alzheimer's disease status, a cognitive decline status and/or a dementia status in a subject. The kit comprises means for determining, in a biological sample obtained from the subject, an amount of at least one multi-antennary protein-derived N-glycan selected from a group consisting of glycan M, glycan R, glycan T, glycan U and glycan. The kit also comprises instructions for predicting the Alzheimer's disease status, the cognitive decline status and/or the dementia status in the subject based on the amount of the at least one multi-antennary protein-derived N-glycan.

Another aspect of the embodiments relates to a kit for evaluating treatment effects of Alzheimer's disease, cognitive decline and/or dementia. The kit comprises means for determining, in a biological sample obtained from a subject treated for Alzheimer disease, cognitive decline and/or dementia, an amount of at least one multi-antennary protein-derived N-glycan selected from a group consisting of glycan M, glycan R, glycan T, glycan U and glycan V. The kit also comprises instructions for evaluating treatment effects of a treatment against Alzheimer's disease, cognitive decline and/or dementia applied to the subject based on the amount of the at least one multi-antennary one protein-derived N-glycan.

### BRIEF DESCRIPTION OF THE DRAWINGS

The embodiments, together with further objectives and advantages thereof, may best be understood by making reference to the following description taken together with the accompanying drawings, in which:

FIG. 1 illustrates mass spectrum of all Asn-linked glycans from the CSF proteome, after enzymatic removal of glycans from glycopeptides, isolation and derivatization of the glycans. Mass spectrometry analysis was done by MALDI/TOF/TOF and a MALDI/TOF mass spectrum is shown. Pooled CSF samples were used from healthy individuals (n=31)

FIG. 2 illustrates mass spectrum of all Asn-linked glycans, analyzed as described in FIG. 1, from the CSF proteome. Pooled CSF samples were used from MCI patients (n=25).

FIG. 3 illustrates mass spectrum of all Asn-linked glycans from the CSF proteome, analyzed as described in FIG. 1. Pooled CSF samples were used from AD patients (n=27).

FIG. 4 illustrates mass spectrum of all Asn-linked glycans, analyzed as described in FIG. 1, using CSF collected from the ventricles of human postmortem brains. Pooled ventricular CSF samples from controls (n=5).

FIG. 5 illustrates mass spectrum of all Asn-linked glycans, analyzed as described in FIG. 1, using CSF collected from the ventricles of human postmortem brains. Pooled ventricular CSF samples from probable AD cases (n=3).

FIG. 6 illustrates mass spectrum of all Asn-linked glycans, analyzed as described in FIG. 1, using CSF collected from the ventricles of human postmortem brains. Pooled ventricular CSF from definitive AD cases (n=5).

### DETAILED DESCRIPTION

The present embodiments generally relate to the usage of protein-derived glycans as biomarkers that can be used for diagnosis and prognosis of various diseases and medical conditions, including Alzheimer's disease (AD), cognitive decline and/or dementia. The protein-derived glycans can also be used in order to evaluate the effect of various treatments against such diseases and medical conditions.

In order to facilitate understanding of the present embodiments definitions of some of the terms used herein follow.

### DEFINITIONS

The term glycan is defined herein as a polysaccharide or oligosaccharide.

The term glycomics is defined herein as the study of all glycans in a given sample. In the embodiments, the glycans studied were derived from glycoproteins. Hence, the term glycomics is used herein to refer to all protein-derived glycans.

The term N-glycomics is defined herein as the study of all asparagine-linked glycans.

The term O-glycomics is defined herein as the study of all glycans that are covalently bound to the OH-group of amino acids (most commonly threonine or serine).

The term terminal sialic acid or sialic acid residue is defined herein as N-acetylneuraminic acid (NeuAc or Neu5Ac).

The terms sialylation and sialylated are used to describe the presence of sialic acid in the glycans concerned.

The term Lewis X epitope or Lewis structure is defined herein as an epitope, within a glycan, containing the trisaccharide sequence Galβ4[Fucα3]GlcNAc, with the Gal residue at the terminal end, wherein Gal denotes galactose, Fuc denotes fucose and GlcNAc denotes N-acetylglucosamine.

The terms bi- and tri-, tetra-, penta- and hexa-antennary glycans are defined herein as glycans that are branched into two, three, four, five or six branches, respectively. The term multi-antennary glycan is defined herein as a glycan comprising multiple, i.e. at least two, branches.

Hence, bi- and tri-, tetra-, penta- and hexa-antennary glycans are examples of multi-antennary glycans.

The term cognitive decline is defined herein as a deterioration in cognitive function. There is a normal process of age related cognitive decline across the life-span characterized by increasing difficulties with memory (new learning), speed of information processing, language and other cognitive functions. This normal process of age related decline is often termed primary ageing. Secondary ageing is the rapid deterioration in function due to a pathological process such as dementia, stroke or acquired brain injury.

The term non-AD dementia is defined herein as all forms of dementia apart from AD.

Disease status and in particular Alzheimer's disease status, cognitive decline status and/or dementia status relates to a status of the relevant disease or medical condition. In an embodiment, the disease status is either defined as suffering or at least likely to suffer from the disease versus not suffering or at least likely not to suffer from the disease. Hence, predicting disease status can, in an embodiment, be implemented as predicting whether a subject is suffering from or at least likely to suffer from Alzheimer's disease, cognitive decline or dementia. In a particular embodiment, predicting Alzheimer's disease status, cognitive decline status and/or dementia status involves predicting whether a subject is likely to suffer from Alzheimer's disease, cognitive decline or dementia or whether the subject is likely to be healthy with regard to Alzheimer's disease, cognitive decline or dementia.

In another embodiment, the disease status could be defined as related to different stages of disease progress such as ranging from an early or mild stage with merely mild symptoms, if any, to late or worse stages with severe symptoms. In this embodiment predicting disease status can be implemented as predicting the relevant Alzheimer's disease stage, the cognitive decline stage or the dementia stage of a subject.

The term surrogate marker is defined herein as a laboratory measurement or physical sign that is used in therapeutic trials as a substitute for a clinically meaningful endpoint that is a direct measure of how a patient feels, functions, or survives and is expected to predict the effect of the therapy.

Specific N-glycans disclosed herein are identified below:

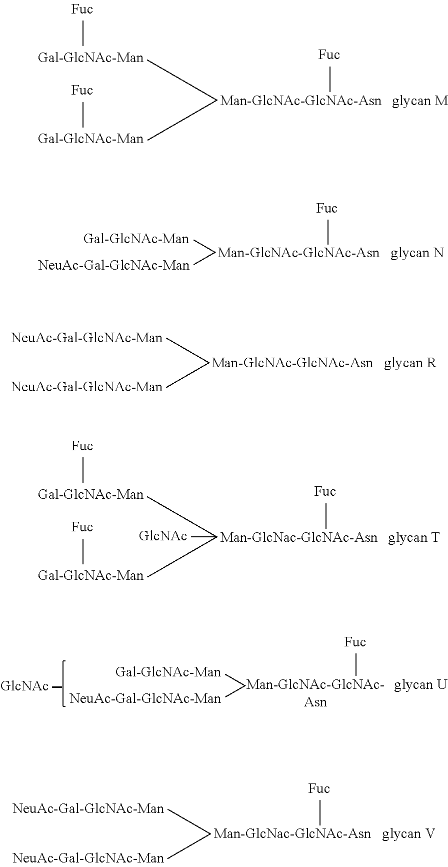

Gal denotes galactose, GlcNAc denotes N-acetylglucosamine, Fuc denotes fucose, Man denotes mannose and NeuAc denotes N-acetylneuraminic acid (also referred to as sialic acid). Asn as shown above indicates the asparagine residue by which the glycan is linked to a protein.

Table 1 below indicates the m/z determined by matrix-assisted laser desorption/ionization-time-of-flight (MALDI/TOF) for the glycans listed above (permethylated glycans).

TABLE 1
m/z for N-glycans

| Glycan | m/z* |
|---|---|
| M | 2592 |
| N | 2605 |
| R | 2792 |
| T | 2837 |
| U | 2850 |
| V | 2966 |

Protein glycosylation is the most common and complex form of posttranslational modification of proteins and plays important biological roles by influencing the roles of glycoproteins, including acting as cellular recognition signals. The biosynthesis of glycans is not controlled by a template but depends on the complicated concerted action of several glycosyltransferases, rendering an almost limitless amount of possible glycan structures. The glycans are generally covalently linked to either an asparagine (N-glycans) or serine/threonine (O-glycans). Glycomics approaches in the search for early biomarkers of AD and related diseases, including cognitive decline and dementia, have not previously been reported, in particular not from cerebrospinal fluid (CSF) samples.

Not many previous reports on glycomics changes in AD exist, with the exception of one report on glycomics changes in AD in serum (Chen 2010). However, that study used serum that was desilylated prior to analysis. Other studies have suggested that the sialic acid-containing isoform of transferrin is decreased in AD.

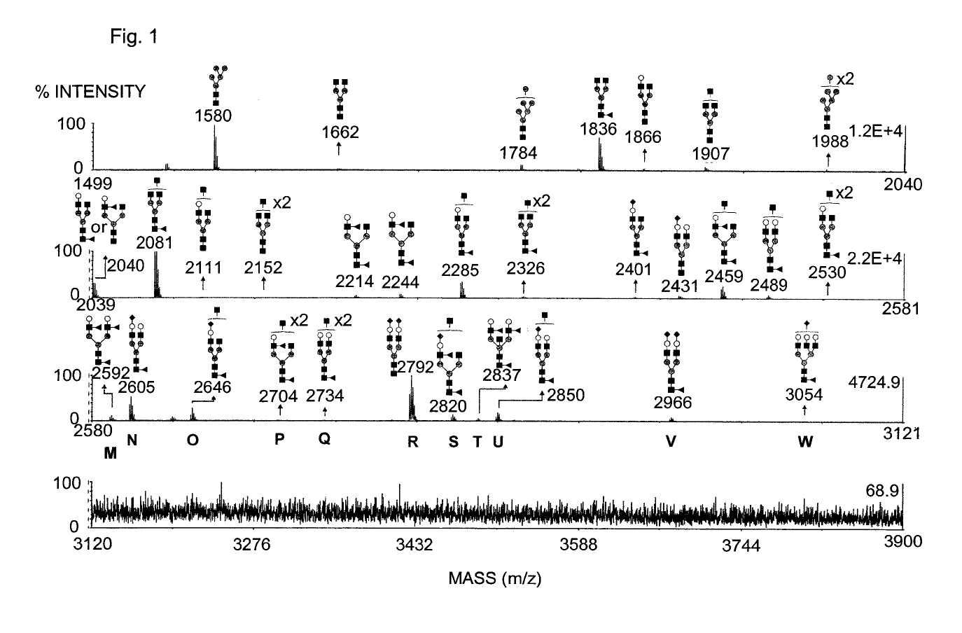

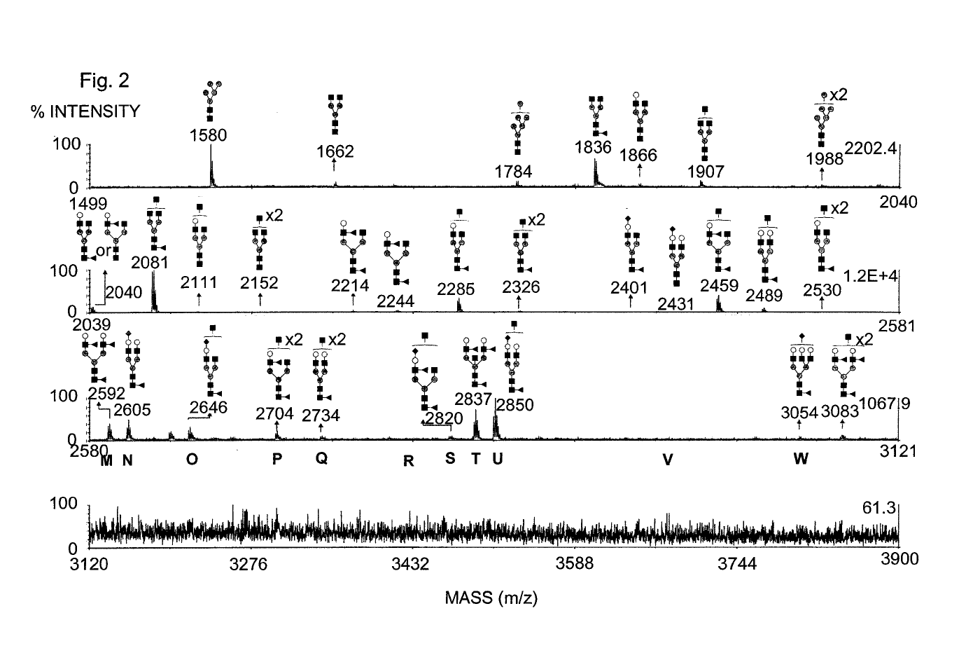

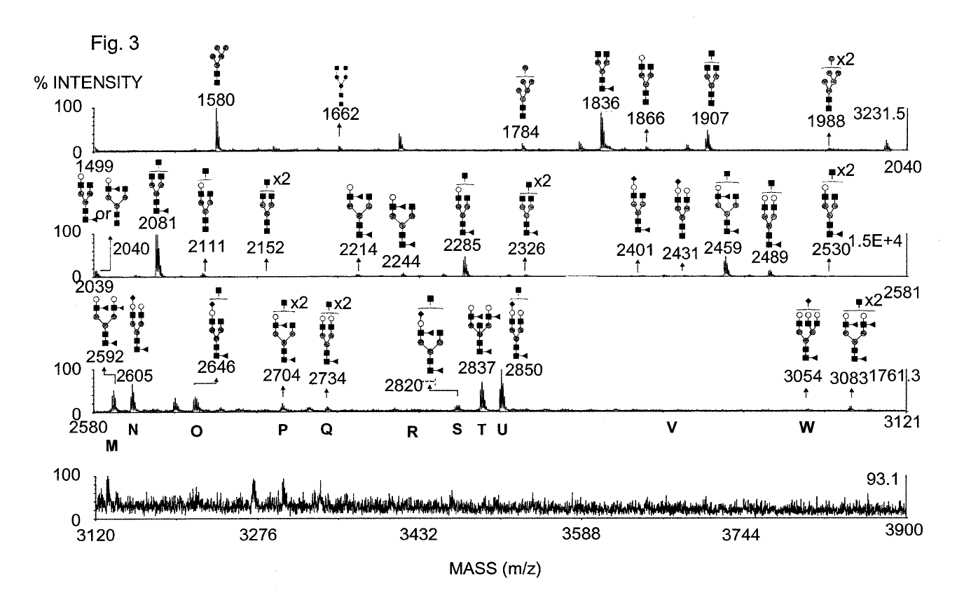

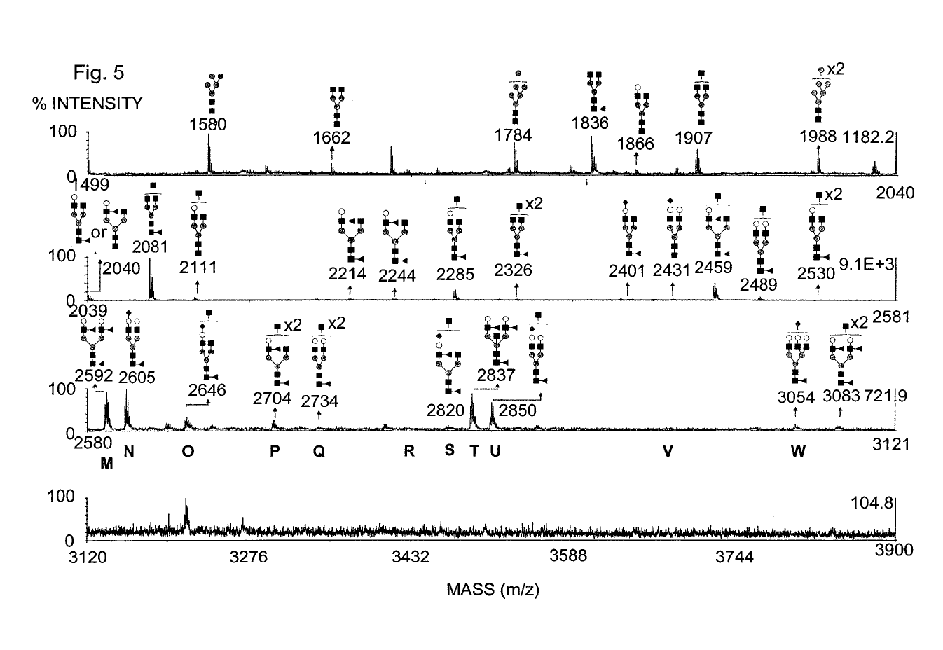

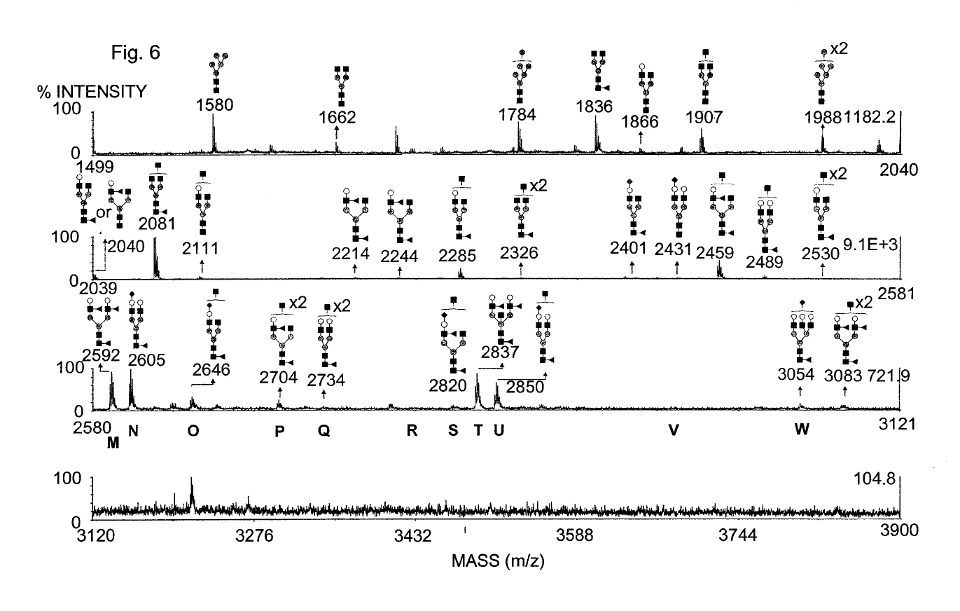

Changes in glycomics, in which full-length glycans are analyzed after their enzymatic removal from all glycoproteins in a given sample, is a novel approach for detecting early, novel biomarkers for AD, dementia and cognitive decline or deficits. Using such an approach, our data has shown several differences in the CSF N-glycome in AD and Mild cognitive impairment (MCI) patients compared to control subjects (FIGS. 1-6). Measuring or determining the amounts of glycans can thus be used as biomarkers for AD/dementia/cognitive decline.

The embodiments relate to monitoring glycomics changes in biological sample from a subject or patient, preferably a mammalian subject or patient and more preferably a human subject or patient. As shown herein, the amounts of such protein-derived glycans from the biological samples, such as CSF samples, from AD, probable AD and MCI or other dementia patients compared to the amounts of the protein-derived glycans in biological samples, such as CSF samples, from control subjects, such as measured by mass spectrometry, can be used as a biomarker for AD/MCI/dementia.

In particular embodiments, all bi- and tri-antennary glycans with two or more terminal sialic acid residues are decreased or even lacking in the MCI, probable AD and AD cases. It is probable that tetra-, penta- and hexaantennary glycans with all antenna capped with sialic acids are also decreased or lacking in the MCI and AD cases. No such tetra-, penta- and hexaantennary glycans were, however, detected in our experiments, because they are less abundant and more difficult to detect with the method used in the examples.

Two glycans, glycans R and V, are decreased or absent in CSF from MCI and AD cases, although they are present in CSF from control cases, see FIGS. 1-6.

Glycans R and V are bi-antennary with two terminal sialic acids and the difference between the two glycans is that glycan V has a fucose attached to the core GlcNAc, whereas glycan R lacks a fucose on the core GlcNAc residue. Bi-antennary glycans carrying a sialic acid residue on only one of the antenna, for instance glycans N and U, are present in both control and AD cases.

Other glycans, for instance peaks M and T in FIG. 1-6, are, in contrast, relatively increased in MCI and AD compared to control cases. These glycans carry Lewis X epitopes. Another glycan that is relatively increased in AD is glycan U, a core fucosylated glycan with one sialic acid.

The more detailed monosaccharide compositions for the respective structures of the protein-derived glycans are shown in FIGS. 1-6 and with the monosaccharide units shown here below:

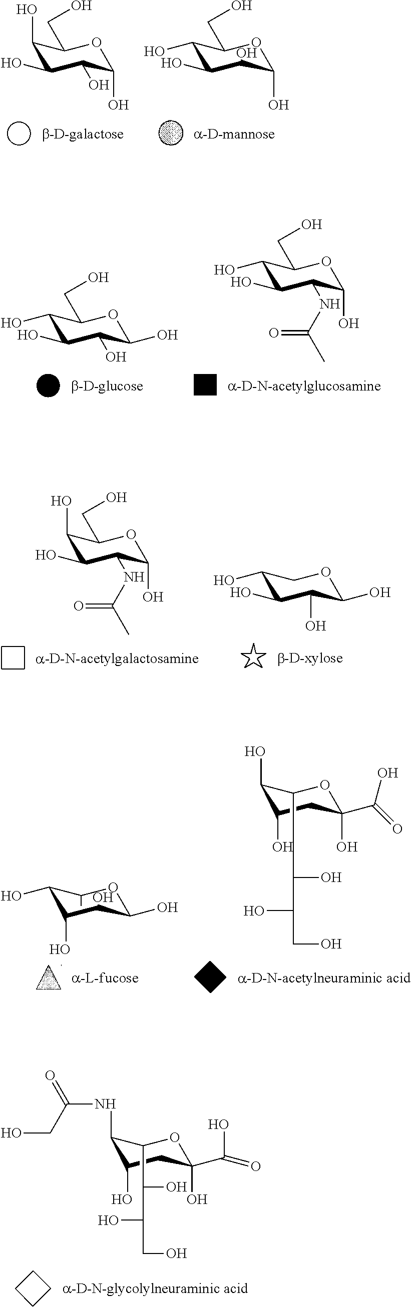

In particular embodiments, the above mentioned analysis of protein-derived glycans is used for prognosis or diagnosis of AD or dementia patients, for instance by measuring the amount of glycans M, R, T, U and/or V or by analyzing the ratio of the amount of at least one glycan that is decreased in MCI and AD cases (glycans R and V) divided by the amount of at least one glycan that is increased in MCI and AD cases (glycans M, T and U) or vice versa, i.e. the amount of glycans that are increased in MCI and AD cases divided by the amount of glycans that are decreased in MCI and AD cases.

In other particular embodiments, the above mentioned analysis is used for monitoring treatment effects during clinical trials for AD or other dementia treatments, for instance by measuring the amount of glycans M, R, T, U and/or V or by analyzing the ratio of the amount of glycans that are increased in MCI and AD cases divided by the amount of glycans that are decreased in MCI and AD cases, or vice versa.

In further particular embodiments, the above mentioned analysis is used for enrichment of the population during clinical trials for AD or other dementia treatments, for instance by measuring the amount of glycans M, R, T, U and/or V or by analyzing the ratio of the amount of glycans that are increased in MCI and AD cases divided by the amount of glycans that are decreased in MCI and AD cases, or vice versa.

In an embodiment, mass spectrometry is used in order to measure or determine the amount of the glycans. An example of a mass spectrometric method that can be used is matrix-assisted laser desorption/ionization-time-of-flight (MALDI/TOF). Another mass spectrometric method that can be used is Ion Trap electrospray ionization (ESI), but still other mass spectrometric techniques are also useful in order to determine an amount of glycans in a biological sample.

The analyses can be done in positive ion mode, which is often used if the glycans have been permethylated prior to analysis but can also be done in negative ion mode, which may be the preferable mode for negatively charged glycans.

To facilitate proteolytic digestion of disulfide-containing glycoproteins, the samples are preferably first subjected to reduction, for instance by dithiotreitol (DTT), to cleave the disulfide bridges and subsequently carboxymethylated in the presence of iodoacetic acid (IAA) to block the disulfides from reforming the disulfide bridges (Dell 1993). The carboxymethylation is terminated, for instance, by dialysis followed by reduction of the volume/drying by lyophilization and/or speedvac. Digestion of the reduced proteins into peptides and glycopeptides is preferably subsequently done by incubation with trypsin, followed by termination of the reaction by boiling and purification by revered phase chromatography (Sun 2008, Sun 2010). N-glycans are preferably subsequently enzymatically removed from glycopeptides, by N-glycosidase F or N-glycosidase A treatment, isolated from O-glycopeptides and other peptides with chromatography, for instance Sep-Pak C18 chromatography.

To facilitate glycan analysis, the samples are preferably subjected to permethylation prior to analysis, for instance using the sodium hydroxide procedure (Dell 1993). Although permethylation has several advantages, including enhancing the detection signal and stabilizing the glycans, it is possible to perform analysis without permethylation. The peptides containing O-linked oligosaccharides obtained after the Sep-Pak C18 chromatography described above can be subjected to reductive elimination in NaBH₄ and NaOH to liberate O-glycopeptides and then desalted through an ion exchange (for instance Dowex 50W-X8) column for further analysis of O-linked glycans (Sun 2008, Sun 2010).

Another technique that can be used to determine the amount of the at least one protein-derived glycan is chromatography, preferably high performance liquid chromatography (HPLC). Sample preparation is preferably done as described for mass spectrometry, as described above, as far as to the N-glycosidase step. The liberated N-glycans are labeled with a light-absorbing group, preferably a fluorescent group, to enable sensitive detection of the glycans by a fluorescence detector. One labeling reaction that is useful for this purpose is reductive amination with 2-aminopyridine (PA) (Ruhaak 2010). The glycans are separated usings columns and gradients according to methods known in the art

Another embodiment for determining the amount of the at least one protein-derived glycan is well plate assays using lectins and/or antibodies that recognize specific glycan epitopes. This assay can be set up in many different combinations. In the bottom layer, plates are coated with antibody or lectin or nothing. In the second layer, the plates are coated with the sample containing the glycans. Glycans are added either as the enzymatically liberated glycans after enzymatic digestion with N-glycosidase F or A, as described for sample preparation prior to mass spectrometric analysis described above (except the permethylation step, which is not needed here). Otherwise, the whole glycan-containing glycoproteomes of the sample are adsorbed to the coated or non-coated plates. In the upper layer, labeled lectins or antibodies are added. Labeling is used in order to generate a signal, which is proportional to the amount of specific glycans within the sample. In an embodiment, biotin-labeled lectins that subsequently can be detected with HRP-conjugated streptavidin are used. In another embodiment, fluorescently labeled lectins are used.

Lectins useful in the context of the present embodiments include lectins that bind to sialic acid, for instance Sambucus Nigra Lectin (SNA), which recognizes sialic acid with a specificity for α-2,6 linked sialic acids and lectins that are specific for α-2,3 linked sialic acid, for instance Maackia Amurensis Lectin II (MAUI). Other lectins that are useful are lectins that recognize GlcNAc and sialic acid, for instance wheat germ agglutinin (WGA). Other lectins that are useful include lectins that are specific for Gal and GlcNac, for instance RCA (Ricinis communis agglutinin) and G Simplicifolia II (GSII). Other lectins that are useful include lectins that bind to mannose, for instance Concanavalin A (Con A). Other lectins that are useful include lectins that bind to the fucosylated core region of bi- and triantennary complex glycans.

Antibodies that are specific for glycan epitopes are also useful. For instance antibodies that are specific for the Lewis X epitope are useful to recognize for instance glycans M and T. By setting up plate assays with a proper combination of lectins and/or antibodies, signals or ratio of signals, for instance for glycans R/(M+T+U) or (R+V)/(M+T), can be generated.

In an embodiment, plate assays with fluorescently labeled lectins and/or antibodies can be used to generate a signal representative of an amount of the at least one protein-derived glycan in terms of measured fluorescence intensity.

In another embodiment, plate assays with fluorescently labeled lectins and/or antibodies can be used to generate a signal representative of an amount of the at least one protein-derived glycan in terms of measured fluorescence resonance energy transfer (FRET).

In an embodiment, fluorescently labeled lectins and/or antibodies together with a glycan-containing biological sample can be used to measure fluorescence signals in a cuvette in a spectrofluorometer.

In another embodiment, fluorescently labeles lectins and/or antibodies together with a glycan-containing biological sample can be used to measure FRET in a cuvette in a spectrophotometer.

Another embodiment relates to the use of a proximity ligation assay (PLA) (Fredriksson 2002) for measuring amounts of protein-derived glycans in the well plate assays described above. In this technique, two detection probes, for instance antibodies, are covalently attached to different DNA strands, which, if they are in proximity, can be ligated and amplified by a rolling circle amplification, allowing highly sensitive detection. For instance by using two different antibodies carrying different DNA strands, against the same epitope, a highly amplifiable signal for the epitope can be generated. Conjugating for instance antibodies specific for Lewis X or lectins recognizing other glycan structures, with oligonucleotides, thus allows development of highly sensitive glycan biomarker assays. Such assays have previously been used to validate, for instance, cancer biomarkers (Fredriksson 2007). Conjugation of antibodies or lectins with DNA strands for proximity ligation assay purposes are commercially available from Olink Bioscience (Uppsala, Sweden).

In an embodiment, the biological sample is preferably treated to separate the protein-derived N-glycans from the proteins before the determination of the amount of the at least one protein-derived N-glycan. Hence, in an embodiment, the determination of the amount is preferably made on N-glycans that are removed from the proteins, i.e. the determination of the amount is preferably made on free N-glycans that are no longer attached to the protein core, and in particular no longer attached to asparagine residues in the protein core.

Although it is generally preferred to analyze the protein-derived N-glycans after they have been separated from their respective core protein, the determination of the amount of the protein-derived N-glycans can, in an embodiment, be performed when the N-glycans are still attached to their respective core protein.

The biological sample is preferably a body fluid sample, such as a CSF sample, a blood sample, a plasma sample, a urine sample, a tear fluid sample, a lymphatic fluid sample or a saliva sample.

In an embodiment, the subject is an animal, such as an experimental animal, e.g. a dog, mouse, rat, fish or a non-human primate. In an embodiment, the experimental animal may be an animal model for a disorder such as Alzheimer disease, dementia or cognitive decline, such as a transgenic, knock-out or knock-in animal. In an embodiment, the subject is a human.

In an embodiment, the method is used for the enrichment of the population in clinical trials for diagnosing patients with Alzheimer's disease, cognitive decline and/or dementia.

In an embodiment, the method is used for a surrogate marker of Alzheimer's disease, cognitive decline and/or dementia during clinical trials. Hence, the at least one multi-antennary protein-derived N-glycan of the embodiments can be monitored and used as a surrogate marker or biomarker during clinical trials. The disease status of the subjects in the clinical trial and/or the treatment effect of a tested treatment, including a drug or medicament, can be monitored using the at least one multi-antennary protein-derived N-glycan.

In an embodiment, the method is used for diagnosing Alzheimer's disease, cognitive decline and/or dementia in a subject, i.e. to determine whether the subject suffers from Alzheimer's disease, cognitive decline and/or dementia.

In an embodiment, the method is used for prognosing Alzheimer's disease, cognitive decline and/or dementia in a subject, i.e. to determine the likelihood or risk that the subject will develop Alzheimer's disease, cognitive decline and/or dementia.

In an embodiment, the method is used for monitoring treatment effects in Alzheimer's disease, cognitive decline and/or dementia in a subject, i.e. to determine whether a particular treatment given to the subject can be expected to be efficient.

An aspect of the embodiments relates to an in vitro method of predicting an Alzheimer's disease status, a cognitive decline status and/or a dementia status in a subject. The in vitro method comprises determining, in a biological sample obtained from the subject, an amount of at least one multi-antennary protein-derived N-glycan in which each antenna is capped with sialic acid. The method also comprises predicting the Alzheimer's disease status, the cognitive decline status and/or the dementia status in the subject based on the amount of the at least one multi-antennary protein-derived N-glycan.

In an embodiment the method further comprises comparing the amount of the at least one multi-antennary protein-derived N-glycan to a respective reference value. The respective reference value could be a corresponding amount of the at least one multi-antennary protein-derived N-glycan measured from at least one, preferably multiple, healthy control subjects. In such a case, the corresponding amount could be an average or median of such amounts of the one multi-antennary protein-derived N-glycan measured from the healthy control subjects. In another embodiment, the respective reference value could be a corresponding amount of the at least one multi-antennary protein-derived N-glycan earlier measured from the particular subject at a time when the subject was confirmed to not suffer from Alzheimer's disease, cognitive decline or dementia.

If the prediction is used to discriminate between different disease stages corresponding amounts of the at least one multi-antennary protein-derived N-glycan can be measured from one or more patient groups in which the patients are diagnosed to be suffering from a particular disease stage. In such a case, multiple reference values could be determined for different groups. For instance, if the amount exceeds a first reference value the subject is predicted to be healthy, if the amount is within an interval defined by the first reference value and a second, smaller reference value the subject is predicted to suffer from a mild or early stage of the particular disease, whereas if the amount is smaller than the second reference value the subject is predicted to suffer from a late or worse stage of the disease. In another example, if the amount is less a first reference value the subject is predicted to be healthy, if the amount is within an interval defined by the first reference value and a second, larger reference value the subject is predicted to suffer from a mild or early stage of the particular disease, whereas if the amount exceeds the second reference value the subject is predicted to suffer from a late or worse stage of the disease. In other embodiment, more disease stages could be used.

In this embodiment the prediction preferably comprises predicting the Alzheimer's disease status, the cognitive decline status and/or the dementia status in the subject based on the comparison of the amount of the at least one multi-antennary protein-derived N-glycan to the respective reference value. Hence, if the amount of the at least one multi-antennary protein-derived N-glycan is higher than or lower than the reference value, depending on which particular multi-antennary protein-derived N-glycan that is monitored, then the subject is predicted to be suffering from Alzheimer's disease, cognitive decline and/or dementia or the subject is predicted to suffer from a late or worse stage of Alzheimer's disease, cognitive decline and/or dementia.

The prediction of the Alzheimer's disease status, the cognitive decline status and/or the dementia status in the subject also encompasses presymptomatic prediction of the Alzheimer's disease status, the cognitive decline status and/or the dementia status and presymptomatic detection of Alzheimer's disease, cognitive decline and/or dementia in a subject.

Another aspect of the embodiments relates to an in vitro method for evaluating treatment effects of Alzheimer's disease, cognitive decline and/or dementia. The in vitro method comprises determining, in a biological sample obtained from a subject treated for Alzheimer's disease, cognitive decline and/or dementia, an amount of at least one multi-antennary protein-derived N-glycan in which each antenna is capped with sialic acid. The method also comprises evaluating treatment effects of a treatment against Alzheimer's disease, cognitive decline and/or dementia applied to the subject based on the amount of the at least one multi-antennary protein-derived N-glycan.

In these embodiments the effect of a particular treatment against Alzheimer's disease, cognitive decline and/or dementia can be evaluated based on whether the amount of at least one multi-antennary protein-derived N-glycan. The particular treatment can be any treatment that is administered or given to the subject and the effect of which should be evaluated. In particular, the treatment could be a drug or medicament, for instance, during a clinical test where the effect of a novel drug or medicament should be tested and evaluated. The treatment could also be one of multiple available alternative treatment regimes. In such a case, the method can be used to identify and select the particular treatment regime(s) that is(are) most effective for the particular subject.

In an embodiment, the method further comprises comparing the amount of the at least one multi-antennary protein-derived N-glycan to a respective reference value. Hence, the treatment can be monitored and determined to be effective or not effective depending on whether the amount exceeds or falls below the respective reference value. In these embodiments, the reference value is preferably a corresponding amount of the at least one multi-antennary protein-derived N-glycan determined prior to the start of the particular treatment. Hence, in a preferred embodiment the subject himself/herself constitutes the control reference. In an alternative embodiment, the reference value is a corresponding amount of the at least one multi-antennary protein-derived N-glycan measured from at least one, preferably multiple, healthy control subjects.

The evaluation of the treatment effects then preferably comprises evaluating the treatment effects of the treatment against Alzheimer's disease, cognitive decline and/or dementia based on the comparison of the amount of the at least one multi-antennary protein-derived N-glycan to the respective reference value.

In an embodiment, an amount of at least one bi- or tri-antennary protein-derived N-glycan in which each antenna is capped with sialic acid is determined in the biological sample. Hence, in this embodiment, the multi-antennary protein-derived N-glycan comprises two or three branches.

In an embodiment, an amount of at least one bi- or tri-antennary protein-derived N-glycan selected from a group consisting of glycan R and glycan V is determined in the biological sample.

In an embodiment, amounts of at least two multi-antennary protein-derived N-glycans are determined in the biological sample. In a particular embodiment, the first multi-antennary protein-derived N-glycan is at least one multi-antennary protein-derived N-glycan having each antenna capped with sialic acid. In a particular embodiment, the second multi-antennary protein-derived N-glycan is at least one multi-antennary protein-derived N-glycan having at least one antenna that lacks sialic acid.

In an embodiment, a ratio of the amounts of the at least two multi-antennary protein-derived N-glycans is calculated. This ratio can then be used, in an embodiment, to predict the Alzheimer's disease status, the cognitive decline status and/or the dementia status in the subject. In another embodiment, the ratio is used to evaluate the treatment effects of the treatment against Alzheimer's disease, cognitive decline and/or dementia.

In an embodiment, an amount of at least one bi- or tri-antennary protein-derived N-glycan selected from a group consisting of glycan R and glycan V and an amount of at least one bi- or tri-antennary protein-derived N-glycan selected from a group consisting of glycan M, glycan T and glycan U are determined in the biological sample.

In a particular embodiment, the ratio calculated between the amounts of the at least two multi-antennary protein-derived N-glycans is selected from a group consisting of:

i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
viii) glycan V/glycan M,
ix) glycan V/glycan T,
x) glycan V/glycan U,
xi) glycan V/(glycan M+glycan T),
xii) glycan V/(glycan M+glycan U),
xiii) glycan V/(glycan T+glycan U),
xiv) glycan V/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to xxi), i.e. ratio b/a if the original ratio was a/b.

In a particular embodiment, the ratio calculated between the amounts of the at least two multi-antennary protein-derived N-glycans is selected from a group consisting of:

i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to vii) and xv) to xxi).

In another particular embodiment, the ratio calculated between the amounts of the at least two multi-antennary protein-derived N-glycans is ratio vii) glycan R/(glycan M+glycan T+glycan U) or an inverse of vii), i.e. (glycan M+glycan T+glycan U)/glycan R.

The ratios i) to xxi) are smaller for a subject suffering from Alzheimer's disease, cognitive decline or dementia as compared to a healthy subject. Correspondingly, the ratios i) to xxi) are smaller for a subject suffering from a late or worse stage of Alzheimer's disease, cognitive decline or dementia as compared to a healthy subject or a subject suffering from an early or mild stage of Alzheimer's disease, cognitive decline or dementia. It therefore follows that the inverse ratios, i.e. xxii) above, are larger for a subject suffering from Alzheimer's disease, cognitive decline or dementia as compared to a healthy subject and are larger for a subject suffering from a late or worse stage of Alzheimer's disease, cognitive decline or dementia as compared to a healthy subject or a subject suffering from an early or mild stage of Alzheimer's disease, cognitive decline or dementia.

The amount of at least one multi-antennary protein-derived N-glycan can be determined, in an embodiment, in the biological sample, using mass spectrometry. In a particular embodiment, the amount is determined using matrix-assisted laser desorption/ionization-time-of-flight (MALDI-TOF).

The amount of at least one multi-antennary protein-derived N-glycan can be determined, in another embodiment, in the biological sample using a well plate assay. In a particular embodiment, the amount is determined using proximity ligation assay (PLA).

In an embodiment, the biological sample is selected from a group consisting of a cerebrospinal fluid sample, a blood sample, a plasma sample, an urine sample, a tear fluid sample, a lymphatic fluid sample or a saliva sample.

In an embodiment, the method involves predicting the Alzheimer's disease status or evaluating treatment effects of Alzheimer's disease.

A further aspect of the embodiments relates to an in vitro method of predicting an Alzheimer's disease status, a cognitive decline status and/or a dementia status in a subject. The method comprises determining, in a biological sample obtained from the subject, an amount of at least one multi-antennary protein-derived N-glycan selected from a group consisting of glycan M, glycan R, glycan T, glycan U and glycan V. The method also comprises predicting the Alzheimer's disease status, the cognitive decline status and/or the dementia status in the subject based on the amount of the at least one multi-antennary protein-derived N-glycan.

Yet another aspect of the embodiments relates to an in vitro method for evaluating treatment effects of Alzheimer's disease, cognitive decline and/or dementia. The in vitro method comprises determining, in a biological sample obtained from a subject treated for Alzheimer's disease, cognitive decline and/or dementia, an amount of at least one multi-antennary protein-derived N-glycan selected from a group consisting of glycan M, glycan R, glycan T, glyan U and glycan V. The method also comprises evaluating treatment effects of a treatment against Alzheimer's disease, cognitive decline and/or dementia applied to the subject based on the amount of the at least one multi-antennary protein-derived N-glycan.

A further aspect of the embodiments relates to kit for performing an in vitro method as disclosed above. A particular embodiment relates to a kit for predicting an Alzheimer's disease status, a cognitive decline status and/or a dementia status in a subject. The kit comprises means for determining, in a biological sample obtained from the subject, an amount of at least one multi-antennary protein-derived N-glycan in which each antenna is capped with sialic acid. The kit also comprises instructions for predicting the Alzheimer's disease status, the cognitive decline status and/or the dementia status in the subject based on the amount of the at least one multi-antennary protein-derived N-glycan. Another particular embodiment relates to a kit for evaluating treatment effects of Alzheimer's disease, cognitive decline and/or dementia. The kit comprises means for determining, in a biological sample obtained from a subject treated for Alzheimer disease, cognitive decline and/or dementia, an amount of at least one multi-antennary protein-derived N-glycan in which each antenna is capped with sialic acid. The kit also comprises instructions for evaluating treatment effects of a treatment against Alzheimer's disease, cognitive decline and/or dementia applied to the subject based on the amount of the at least multi-antennary one protein-derived N-glycan.

Yet another aspect of the embodiments relates to a kit for predicting an Alzheimer's disease status, a cognitive decline status and/or a dementia status in a subject. The kit comprises means for determining, in a biological sample obtained from the subject, an amount of at least one multi-antennary protein-derived N-glycan selected from a group consisting of glycan M, glycan R, glycan T, glycan U and glycan. The kit also comprises instructions for predicting the Alzheimer's disease status, the cognitive decline status and/or the dementia status in the subject based on the amount of the at least one multi-antennary protein-derived N-glycan.

Another aspect of the embodiments relates to a kit for evaluating treatment effects of Alzheimer's disease, cognitive decline and/or dementia. The kit comprises means for determining, in a biological sample obtained from a subject treated for Alzheimer disease, cognitive decline and/or dementia, an amount of at least one multi-antennary protein-derived N-glycan selected from a group consisting of glycan M, glycan R, glycan T, glycan U and glycan V. The kit also comprises instructions for evaluating treatment effects of a treatment against Alzheimer's disease, cognitive decline and/or dementia applied to the subject based on the amount of the at least one multi-antennary one protein-derived N-glycan.

The particular means for determining the amount in the kit depends on the particular measurement technique employed in order to determine the amount of the at least one multi-antennary protein-derived N-glycan.

For instance, in the case of MALDI/TOF the means could include reagents employed to prepare the glycans, such as the previously mentioned DTT; carboxymethyl; IAA; trypsin; N-glycosidase F and/or A; Sep-Pak C18 chromograph; optionally NaBH₄ and/or NaOH.

For instance, in the case of HPLC the means could include at least some of the reagents mentioned above for MALDI/TOF in addition to a fluorescent label, such as PA.

For instance, in the case of well plate assays the means could include lectins, such as biotin-labeled lectins, fluorescently labeled lectins, and/or antibodies that recognize specific glycan epitopes; a plate with at least one reaction well or a cuvette; optionally at least some of the reagents mentioned above for MALDI/TOI; optionally HRP-conjugated steptavidin. Non-limiting examples of lectins that can be used in this embodiment have previously been disclosed herein.

### EXAMPLES

### Example 1

Patient and control CSF samples were taken from the Karolinska University Hospital in Huddinge (Sweden). CSF samples were collected by lumbar puncture through the L3/L4 or L4/L5 interspace after local anesthetic infiltration in the skin. After disposal of the first milliliter, CSF was collected in polypropylene tubes, mixed to avoid gradient effects and centrifuged to 2000×g for 10 min to eliminate cells and insoluble material. Supernatants were immediately aliquoted, frozen and stored at −80° C. Samples were tested for albumin ratio, IgG index and cell counts and only samples with normal levels were included in the study. Three pools of CSF samples from two patient groups were used in this study and relatives of the patients were normally used as control subjects, comprising; control (n=31), stable MCI (n=25) and AD (n=27) patients. For glycan structure determination, CSF samples were subjected to reduction, carboxymethylation and tryptic digestion. N-glycans were enzymatically removed from glycopeptides by N-glycosidase F treatment, isolated from O-glycopeptides and other peptides with Sep-Pak C18 chromatography, and subjected to permethylation with the sodium hydroxide procedure, prior to MS analysis (Sun et al, 2008, Sun et al, 2010).

MS and MS/MS data were acquired on a MALDI-TOF/TOF, using an Ultra-flex TOF/TOF instrument (Bruker), as described previously (Sun et al, 2008, Sun et al, 2010). The mass spectra comprising all glycans analyzed are shown in FIGS. 1-3.

The alterations in the relative amounts of the glycans mentioned above are apparent by measuring the ratio between different glycans. The ratios between these peaks have been calculated for the samples shown in FIG. 1-3 (Table 2). The ratio R/(M+T+U) is at least 100-fold lower in MCI and AD as compared control CSF.

TABLE 2
Relative amounts of selected glycans
Peak

| | M | N | R | T | U | V | R/(M + T + U) |
|---|---|---|---|---|---|---|---|
| Cntr | 7 | 31 | 58 | 4 | 11 | 5 | 2.64 |
| MCI | 20 | 24 | 2 | 35 | 48 | 2 | 0.019 |
| AD | 28 | 35 | 2 | 37 | 53 | 2 | 0.017 |

### Example 2

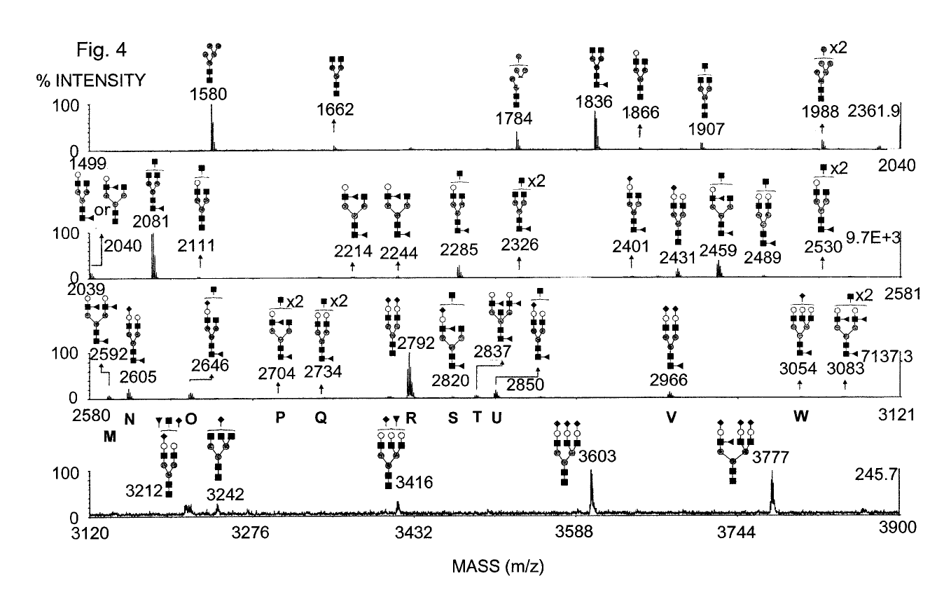

Human postmortem brains were taken from the Huddinge Brain Bank at the Karolinska University Hospital in Huddinge (Sweden) and stored at −80° C. before use. All definitive AD cases, which were sporadic cases, met the critera for definitive AD according to the Consortium to Establish a Registry for AD (Bogdanivic and Morris 1995, Mirra S S et al 1991). The control subjects had no known symptoms of neurological or psychiatric disorders. Control, probable AD and definitive AD cases were age-matched in terms of age, gender and postmortem times, which varied between 9 and 36 hours. CSF from three groups of pooled cases comprising controls (n=5), probable AD (n=3) and definitive AD (n=5) were analyzed in the same manner as described in Example 1. The mass spectra comprising all glycans analyzed are shown in FIGS. 4-6 and the results are presented in Table 3 below.

TABLE 3
Relative amounts of selected glycans
Peak

| | M | N | R | T | U | V | R/(M + T + U) |
|---|---|---|---|---|---|---|---|
| Cntr | 5 | 12 | 53 | 5 | 10 | 7 | 3.1 |
| proAD | 46 | 50 | 4 | 49 | 33 | 3 | 0.031 |
| defAD | 38 | 4 | 4 | 50 | 3 | 3 | 0.043 |

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

Doecke et al (2012) Arch Neurol. 2012 Jul. 16:1-8. doi: 10.1001/archneurol.2012.1282. [Epub ahead of print]
O'Bryant et al (2011) PLoS One, 2011; 6(12):e28092. doi: 10.1371/journal.pone.0028092.
Chen et al (2010) Rejuvenation research, 13: 439-444
Sun et al (2008) J Biol Chem, 283: 18601-18611.
Sun et al (2010) Biochim Biophys Res Comm, 403: 198-202.
Dell et al (1993) Glycobiology: A practical approach, pp 187-222, Oxford University Press, Oxford.
Ruuhaak et al (2010) Anal Bioanal Chem 397:3457-3481.
Bogdanovic and Morris (1995) In: Cruz-Sanchez F F, Ravid R, Cuzner M L, editors. Neuropathological diagnostic critera for brain banking. Amsterdam: IOS Press. P 20-29.
Mirra et al (1991) Neurology 41: 479-486.
Fredriksson et al (2002) Nat Biotechnol 20: 473-477.
Fredriksson et al (2007) Nat Methods 4:327-329.

1-24. (canceled)

25. A method of guiding a subject's treatment for Alzheimer's disease, cognitive decline and/or dementia, comprising:
(a) measuring, in a biological sample obtained from said subject, an amount of at least one bi- or tri-antennary protein derived N-glycan in which each antenna is capped with sialic acid and/or an amount of at least one bi- or tri-antennary protein derived N-glycan that has at least one antenna that lacks sialic acid;
(b) administering a treatment to said subject;
(c) measuring, in a biological sample obtained from said subject, an amount of at least one bi- or tri-antennary protein derived N-glycan in which each antenna is capped with sialic acid and/or an amount of at least one bi- or tri-antennary protein derived N-glycan that has at least one antenna that lacks sialic acid;
(d) comparing the amount of said at least one bi- or tri-antennary protein derived N-glycan measured in step (a) with the amount measured in step (c), wherein a decrease in the amount of said at least one bi- or tri-antennary protein derived N-glycan in which each antenna is capped with sialic acid and/or an increase in the amount of at least one bi- or tri-antennary protein derived N-glycan that has at least one antenna that lacks sialic acid measured in step (c) compared to the amount of the same N-glycan measured in step (a) means the treatment is not effective and the treatment is discontinued or altered, and an increase in the amount of said at least one bi- or tri-antennary protein derived N-glycan in which each antenna is capped with sialic acid and/or a decrease in the amount of at least one bi- or tri-antennary protein derived N-glycan that has at least one antenna that lacks sialic acid measured in step (c) compared to the amount of the same N-glycan measured in step (a) means the treatment is effective and the treatment is continued, thereby guiding a subject's treatment for Alzheimer's disease, cognitive decline and/or dementia.

26. The method of claim 25, wherein the at least one bi- or tri-antennary protein derived N-glycan in which each antenna is capped with sialic acid is glycan R and/or glycan V and the at least one bi- or tri-antennary protein derived N-glycan that has at least one antenna that lacks sialic acid is glycan M, glycan T, and/or glycan U:

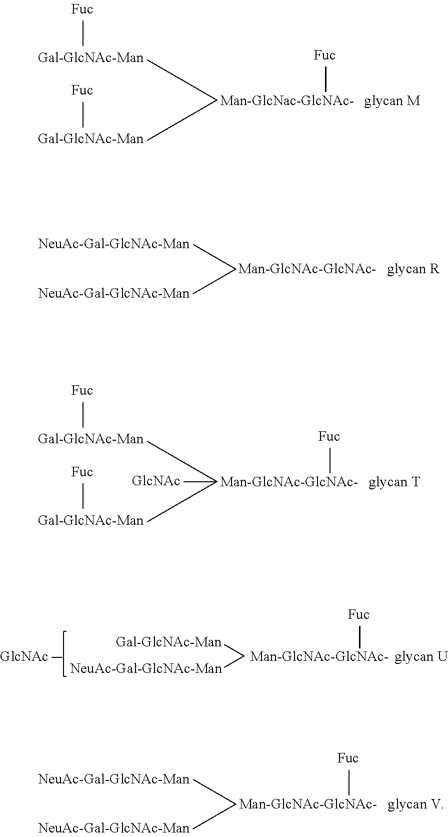

27. A method of guiding a subject's treatment for Alzheimer's disease, cognitive decline and/or dementia, comprising:
(a) (i) measuring, in a biological sample obtained from said subject, an amount of at least one bi- or tri-antennary protein derived N-glycan in which each antenna is capped with sialic acid, wherein the N-glycan is selected from glycan R, glycan V, and any combination thereof: 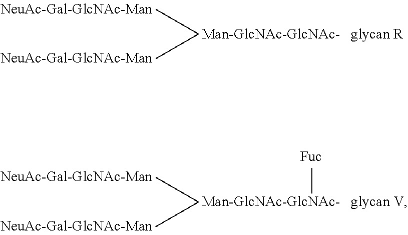
and
(ii) measuring in said biological sample obtained from said subject (of (a) (i)), an amount of at least one bi- or tri-antennary protein derived N-glycan that has at least one antenna that lacks sialic acid, wherein the N-glycan is selected from glycan M, glycan T, glycan U, and any combination thereof: 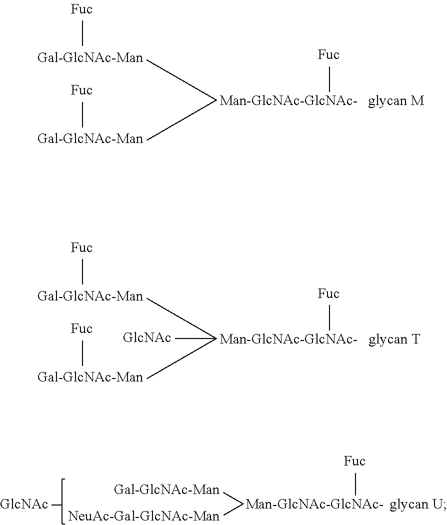
(b) calculating a ratio of said amounts measured in step (a) selected from the group consisting of
i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
viii) glycan V/glycan M,
ix) glycan V/glycan T,
x) glycan V/glycan U,
xi) glycan V/(glycan M+glycan T),
xii) glycan V/(glycan M+glycan U),
xiii) glycan V/(glycan T+glycan U),
xiv) glycan V/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U),
xxii) any inverse of i) to xxi) and any combination thereof;
(c) administering a treatment to said subject;
(d) (i) measuring, in a biological sample obtained from said subject, an amount of said at least one bi- or tri-antennary protein derived N-glycan in which each antenna is capped with sialic acid, wherein the N-glycan is selected from glycan R, glycan V, and any combination thereof, and (ii) measuring in said biological sample obtained from said subject (of (d) (i)), an amount of at least one bi- or tri-antennary protein derived N-glycan that has at least one antenna that lacks sialic acid, wherein the N-glycan is selected from glycan M, glycan T, glycan U:
(e) calculating a ratio of said amounts measured in step (d) selected from the group consisting of:
i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
viii) glycan V/glycan M,
ix) glycan V/glycan T,
x) glycan V/glycan U,
xi) glycan V/(glycan M+glycan T),
xii) glycan V/(glycan M+glycan U),
xiii) glycan V/(glycan T+glycan U),
xiv) glycan V/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to xxi).
(f) comparing the ratios calculated in step (b) to the ratios calculated in step (e), wherein a decrease in the ratio calculated in step (e) as compared to step (b) means the treatment is not effective, and the treatment is discontinued or altered, and an increase in the ratio calculated in step (e) as compared to step (b) means the treatment is effective and the treatment is continued, thereby guiding a subject's treatment for Alzheimer's disease, cognitive decline and/or dementia.

28. The method of claim 27, wherein the ratio calculated in step (b) or step (d) is selected from the group consisting of:
i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to vii) and xv) to xxi).

29. The method according to claim 27, wherein the ratio calculated in step (b) or step (e) is: (i) glycan R/(glycan M+glycan T+glycan U) and/or the inverse of glycan R/(glycan M+glycan T+glycan U) and/or (ii) glycan R/glycan T and/or the inverse of glycan R/glycan T.

30. The method according to claim 25, wherein measuring said amount comprises determining, in said biological sample, said amount of at least one multi-antennary protein-derived N-glycan using mass spectrometry, optionally matrix-assisted laser desorption/ionization-time-of-flight (MALDI-TOF), a well plate assay, high pressure liquid chromatography (HPLC), or a proximity ligation assay (PL A).

31. The method according to claim 27, wherein measuring said amount comprises determining, in said biological sample, said amount of at least one multi-antennary protein-derived N-glycan using mass spectrometry, optionally matrix-assisted laser desorption/ionization-time-of-flight (MALDI-TOF), a well plate assay, high pressure liquid chromatography (HPLC) or a proximity ligation assay (PL A).

32. The method according to claim 25, wherein the biological sample is selected from a group consisting of a cerebrospinal fluid sample, a blood sample, a plasma sample, an urine sample, a tear fluid sample, a lymphatic fluid sample, a saliva sample, and any combination thereof.

33. The method according to claim 27, wherein the biological sample is selected from a group consisting of a cerebrospinal fluid sample, a blood sample, a plasma sample, an urine sample, a tear fluid sample, a lymphatic fluid sample, a saliva sample, and any combination thereof.

34. A method of determining the efficacy of a treatment for Alzheimer's disease, cognitive decline and/or dementia, comprising:
(a) measuring, in a biological sample obtained from said subject, an amount of at least one bi- or tri-antennary protein derived N-glycan in which each antenna is capped with sialic acid and/or an amount of at least one bi- or tri-antennary protein derived N-glycan that has at least one antenna that lacks sialic acid;
(b) administering a treatment to said subject;
(c) measuring, in a biological sample obtained from said subject, an amount of at least one bi- or tri-antennary protein derived N-glycan in which each antenna is capped with sialic acid and/or an amount of at least one bi- or tri-antennary protein derived N-glycan that has at least one antenna that lacks sialic acid;
(d) comparing the amount of said at least one bi- or tri-antennary protein derived N-glycan measured in step (a) with the amount measured in step (c), wherein a decrease in the amount of said at least one bi- or tri-antennary protein derived N-glycan in which each antenna is capped with sialic acid and/or an increase in the amount of at least one bi- or tri-antennary protein derived N-glycan that has at least one antenna that lacks sialic acid measured in step (c) compared to the amount of the same N-glycan measured in step (a) means the treatment is not effective, and an increase in the amount of said at least one bi- or tri-antennary protein derived N-glycan in which each antenna is capped with sialic acid and/or a decrease in the amount of at least one bi- or tri-antennary protein derived N-glycan that has at least one antenna that lacks sialic acid measured in step (c) compared to the amount of the same N-glycan measured in step (a) means the treatment is effective, thereby determining the efficacy of said treatment for Alzheimer's disease, cognitive decline and/or dementia.

35. The method of claim 34, wherein the at least one bi- or tri-antennary protein derived N-glycan in which each antenna is capped with sialic acid is glycan R and/or glycan V and the at least one bi- or tri-antennary protein derived N-glycan that has at least one antenna that lacks sialic acid is glycan M, glycan T, and/or glycan U:

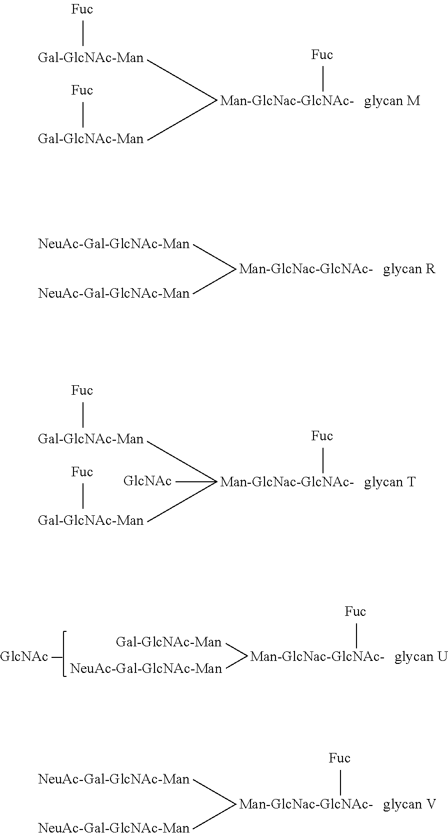

36. A method of determining the efficacy of a treatment for Alzheimer's disease, cognitive decline and/or dementia, comprising:
(a) (i) measuring, in a biological sample obtained from a subject, an amount of at least one bi- or tri-antennary protein derived N-glycan in which each antenna is capped with sialic acid, wherein the N-glycan is selected from glycan R, glycan V, and any combination thereof: 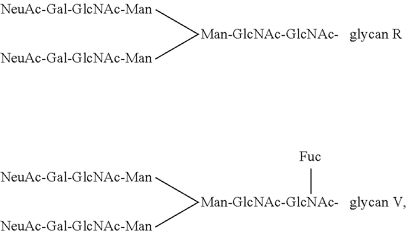
(ii) measuring in said biological sample obtained from said subject (of (a) (i)), an amount of at least one bi- or tri-antennary protein derived N-glycan that has at least one antenna that lacks sialic acid, wherein the N-glycan is selected from glycan M, glycan T, glycan U, and any combination thereof: 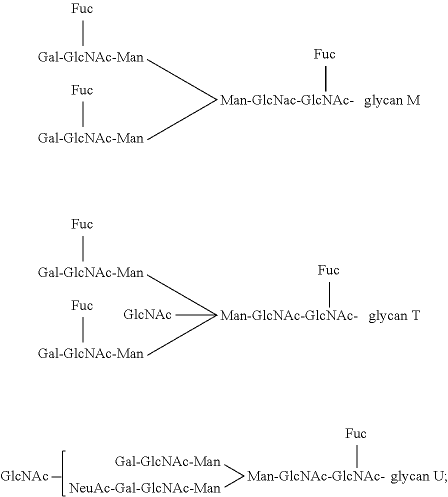
(b) calculating a ratio of said amounts measured in step (a) selected from the group consisting of
i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
viii) glycan V/glycan M,
ix) glycan V/glycan T,
x) glycan V/glycan U,
xi) glycan V/(glycan M+glycan T),
xii) glycan V/(glycan M+glycan U),
xiii) glycan V/(glycan T+glycan U),
xiv) glycan V/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to xxi).
(c) administering a treatment to said subject;
(d) (i) measuring, in a biological sample obtained from said subject, an amount of said least one bi- or tri-antennary protein derived N-glycan in which each antenna is capped with sialic acid, wherein the N-glycan is selected from glycan R, glycan V, and any combination thereof, and (ii) measuring in said biological sample obtained from said subject (of (d) (i)), an amount of at least one bi- or tri-antennary protein derived N-glycan that has at least one antenna that lacks sialic acid, wherein the N-glycan is selected from glycan M, glycan T, glycan U:
(e) calculating a ratio of said amounts measured in step (d) selected from the group consisting of:
i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
viii) glycan V/glycan M,
ix) glycan V/glycan T,
x) glycan V/glycan U,
xi) glycan V/(glycan M+glycan T),
xii) glycan V/(glycan M+glycan U),
xiii) glycan V/(glycan T+glycan U),
xiv) glycan V/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to xxi).
(f) comparing the ratios calculated in step (b) to the ratios calculated in step (e), wherein a decrease in the ratio calculated in step (e) as compared to step (b) means the treatment is not effective, and an increase in the ratio calculated in step (e) as compared to step (b) means the treatment is effective, thereby determining the efficacy of said treatment for Alzheimer's disease, cognitive decline and/or dementia.

37. The method of claim 36, wherein the ratio calculated in step (b) or step (e) is selected from the group consisting of:
i) glycan R/glycan M,
ii) glycan R/glycan T,
iii) glycan R/glycan U,
iv) glycan R/(glycan M+glycan T),
v) glycan R/(glycan M+glycan U),
vi) glycan R/(glycan T+glycan U),
vii) glycan R/(glycan M+glycan T+glycan U),
xv) (glycan R+glycan V)/glycan M,
xvi) (glycan R+glycan V)/glycan T,
xvii) (glycan R+glycan V)/glycan U,
xviii) (glycan R+glycan V)/(glycan M+glycan T),
xix) (glycan R+glycan V)/(glycan M+glycan U),
xx) (glycan R+glycan V)/(glycan T+glycan U),
xxi) (glycan R+glycan V)/(glycan M+glycan T+glycan U), and
xxii) any inverse of i) to vii) and xv) to xxi).

38. The method according to claim 36, wherein the ratio calculated in step (b) or step (d) is: (i) glycan R/(glycan M+glycan T+glycan U) and/or the inverse of glycan R/(glycan M+glycan T+glycan U) and/or (ii) glycan R/glycan T and/or the inverse of glycan R/glycan T.

39. The method according to claim 34, wherein measuring said amount comprises determining, in said biological sample, said amount of at least one multi-antennary protein-derived N-glycan using mass spectrometry, optionally matrix-assisted laser desorption/ionization-time-of-flight (MALDI-TOF), a well plate assay, high pressure liquid chromatography (HPLC), or a proximity ligation assay (PL A).

40. The method according to claim 36, wherein measuring said amount comprises determining, in said biological sample, said amount of at least one multi-antennary protein-derived N-glycan using mass spectrometry, optionally matrix-assisted laser desorption/ionization-time-of-flight (MALDI-TQF), a well plate assay, high pressure liquid chromatography (HPLC), or a proximity ligation assay (PL A).

41. The method according to claim 34, wherein the biological sample is selected from a group consisting of a cerebrospinal fluid sample, a blood sample, a plasma sample, an urine sample, a tear fluid sample, a lymphatic fluid sample, a saliva sample, and any combination thereof.

42. The method according to claim 36, wherein the biological sample is selected from a group consisting of a cerebrospinal fluid sample, a blood sample, a plasma sample, an urine sample, a tear fluid sample, a lymphatic fluid sample saliva sample, and any combination thereof.

43. A method of enriching a population for a clinical trial for Alzheimer's disease, cognitive decline and/or dementia, comprising:
(a) measuring, in a biological sample obtained from said subject, an amount of at least one bi- or tri-antennary protein derived N-glycan in which each antenna is capped with sialic acid and/or an amount of at least one bi- or tri-antennary protein derived N-glycan that has at least one antenna that lacks sialic acid;
(b) comparing the amount of said at least one bi- or tri-antennary protein derived N-glycan measured in step (a) with a reference value, wherein a decrease in the amount of said at least one bi- or tri-antennary protein derived N-glycan in which each antenna is capped with sialic acid and/or an increase in the amount of at least one bi- or tri-antennary protein derived N-glycan that has at least one antenna that lacks sialic acid measured in step (a) compared to said reference value means the subject is not selected for the clinical trial, and an increase in the amount of said at least one bi- or tri-antennary protein derived N-glycan in which each antenna is capped with sialic acid and/or a decrease in the amount of at least one bi- or tri-antennary protein derived N-glycan that has at least one antenna that lacks sialic acid measured in step (a) compared to said reference value means the subject is selected for the clinical trial; and
(c) selecting subjects for said clinical trial having an increase in the amount of said at least one bi- or tri-antennary protein derived N-glycan in which each antenna is capped with sialic acid and/or a decrease in the amount of at least one bi- or tri-antennary protein derived N-glycan that has at least one antenna that lacks sialic acid measured in step (a) compared to said reference value, thereby enriching a population for a clinical trial for Alzheimer's disease, cognitive decline and/or dementia.

44. The method of claim 43, wherein the at least one bi- or tri-antennary protein derived N-glycan in which each antenna is capped with sialic acid is glycan R and/or glycan V and the at least one bi- or tri-antennary protein derived N-glycan that has at least one antenna that lacks sialic acid is glycan M, glycan T, and/or glycan U: [chemical structure]
